(19) United States
(12) Patent Application Publication
(10) Pub. No.: US 2004/0133195 A1
(43) Pub. Date: Jul. 8, 2004

(54) Percutaneous surgical instruments and methods using the same

(76) Inventor: Stephen Solomon, Baltimore, MD (US)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/625,079

(22) Filed: Jul. 22, 2003

Related U.S. Application Data
(60) Provisional application No. 60/397,870, filed on Oct. 3, 2002.

(51) Int. Cl. A61B 18/14

(52) U.S. Cl. 606/41; 606/49

(57) ABSTRACT

Method for ablating a tumor in a body while protecting a nearby structure from the effects of the ablation in which an ablation shield is interposed between the tumor and the nearby structure and then one or more ablation sources of an ablation device is activated to ablate the tumor while the nearby structure is shielded by the ablation shield. Preferably, the ablation procedure is performed while imaging at least a region including and surrounding the tumor so that the ablation device can be guided toward the tumor based on the imaging. The ablation shield can also be guided percutaneously between the nearby structure and the tumor based on the imaging. Additional surgical instruments for percutaneous use and method for performing surgery using the same are also disclosed.

### CROSS REFERENCE TO RELATED APPLICATION

This application claims priority under 35 U.S.C. §119(e) of U.S. provisional patent application Ser. No. 60/397,870 filed Jul. 22, 2002.

### FIELD OF THE INVENTION

The present invention relates generally to percutaneous surgical instruments and more particularly to surgical instruments which prevent damage to an organ or tissue in a body during ablation or radiation of a nearby tumor.

The present invention also relates generally to a method and apparatus for identifying tools and instruments used during surgery which is imaged and more particularly to a method and apparatus for enabling the identification of different tools used during a surgery in which multiple tools are placed in the body simultaneously.

The present invention also relates to a method and apparatus for enabling electrocautery percutaneously.

The present invention further relates to a method for marking or labeling a treatment zone after percutaneous ablation of a tumor or a similar surgical procedure.

The present invention also relates to a method and apparatus for performing an appendectomy or similar surgical procedure in which a single device is capable of performing the required surgical steps.

### BACKGROUND OF THE INVENTION

Advances in imaging have allowed many surgical procedures to become percutaneous ones, i.e., able to be perform through the skin. Incisions in surgery are performed in order to allow visualization of structures via cameras and other optical imaging devices. Although laparoscopic surgery requires insufflation in order to enable visualization, advances in imaging technology with CT, MR, ultrasound, nuclear medicine and x-rays allow a physician to “see” through the skin to perform surgical procedures without having to make incisions in the skin. The elimination of incisions in the skin for at least some surgical procedures reduces the recuperation time and the risk of possible infections.

One common procedure performed percutaneously which utilizes imaging, rather than surgical incision and direct visualization, is the ablation of tumors with percutaneous needle devices. The imaging system guides the placement of the percutaneous needle device into the tumor for ablation. Such procedures are being used more commonly today and their use is expected to increase.

The most common of the percutaneous needle devices relies on radiofrequency energy, but microwave, laser, ultrasound, cryotherapy, and interstitial irradiation are similar means for performing tumor ablation. Radiofrequency ablation is used for treating localized cancer tumors that cannot be removed surgically, such as liver tumors. Liver tumors are usually located deep inside the body where surgeons cannot reach with their hands easily.

In a common radiofrequency ablation procedure, the first step is to place a needle with a plastic insulated shaft into the tumor with imaging guidance. Then, the surgeon presses a button on the needle probe and wires similar to the backbone of an umbrella shoot out and cover a portion or all of the tumor. Once the patient is connected to ground, alternating radiofrequency current from the wire tips then agitate ions in the tissue surrounding the needle, creating frictional heat to heat the tumor. The radiofrequency current thus serves as an ablation source for enabling ablation of the tumor.

A few clinical examples of radiofrequency ablation of tumors include radiofrequency ablation of renal tumors (see Su L M, Jarrett T. W., Chan D. Y., Kavoussi L. R., Solomon S. B., Percutaneous computed tomography-guided radiofrequency ablation of renal masses in high surgical risk patients: preliminary results. Urology 2003 April; 61(4 Supplement 1):26-33), laser ablation of liver tumors (see Dick E. A., Joarder R, de Jode M, Taylor-Robinson S. D., Thomas H. C., Foster G. R., Gedroyc W. M., MR-guided laser thermal ablation of primary and secondary liver tumours. Clin. Radiology 2003 February; 58(2):112-20, and Erce C, Parks R. W., Interstitial ablative techniques for hepatic tumours. Br J Surg 2003 March; 90(3):272-89), and radiofrequency ablation of adrenal tumors (see Wood B. J., Abraham J, Hvizda J. L., Alexander H. R., Fojo T., Radiofrequency ablation of adrenal tumors and adrenocortical carcinoma metastases. Cancer 2003 February 1;97(3):554-60).

One of the major limitations to this type of tumor ablation procedure is the lack of tools to optimize these procedures. For example, when critical healthy, normal structures, such as organs and tissue, are near targeted cancerous structures, the critical ones are liable to be inadvertently, detrimentally affected and damaged by the thermal or radiation heat being released by the ablation needle probe and applied to the target structures. It would be desirable to move these critical normal organs and tissue away from the targeted cancerous structures because it has been documented that complications to normal tissue may occur when ablating a nearby tumor (see Chopra S, Dodd G. D. 3rd, Chanin M. P., Chintapalli K N., Radiofrequency ablation of hepatic tumors adjacent to the gallbladder: feasibility and safety. AJR Am J Roentgenol 2003 March; 180(3):697-701). For example, in placing a percutaneous needle, a loop of bowel might be retracted away from the site of the needle tip. Another example would be when applying energy for thermal ablation or interstitial radiation, one might want to separate normal tissue from the ablation source. Ideally, there would be thermal or radiation protection devices that could shield the normal tissue or structures from the deadly thermal or radiation energy applied.

In a separate but related field, when a biopsy is performed, a portion of a structure is removed possibly causing bleeding from a remaining part of the structure. There is not believed to be a tool which can be guided by images to stop bleeding percutaneously.

In laparoscopic surgery, incisions are smaller because visualization is performed through small cameras or other imaging devices. While these cameras provided remote visualization, they required a new set of tools to enable the procedures (see Seifman B. D., Wolf J. S. Jr., Technical advances in laparoscopy: hand assistance, retractors, and the pneumodissector. J, Endourol. 2000 Dec. 14(10):921-8). Retractors and cautery devices are examples of devices that had to be specifically made for this new type of surgery.

### OBJECTS AND SUMMARY OF THE INVENTION

It is an object of the present invention to provide a new method for ablating tumors while shielding surrounding normal structures, such as tissue and organs.

It is another object of the present invention to provide new apparatus for placement between a cancerous structure and surrounding normal structures to shield the normal structures from the application of treatment to the cancerous structure, such as the application of heat, cold, radiation and ultrasound, and methods for using the same.

It is another object of the present invention to provide new tools for distancing healthy tissue and organs from a cancerous tumor being treated by ablation, in particular tools capable of being used percutaneously and guided by imaging techniques to positions between the tumor and the healthy tissue and organs, and methods for using the same.

It is yet another object of the present invention to provide a method and apparatus for identifying tools and instruments used during surgery which is imaged and more particularly to a method and apparatus for enabling the identification of different tools used during a surgery in which multiple tools are placed in the body simultaneously.

Another object of the present invention is to provide a new method and apparatus for enabling electrocautery percutaneously.

Yet another object of the present invention is to provide a new method and apparatus for marking or labeling a treatment zone after percutaneous ablation of a tumor or a similar surgical procedure.

Still another object of the present invention also relates to a new method and apparatus for performing an appendectomy or similar surgical procedure in which a single device is capable of performing the required surgical steps.

In order to achieve at least some of the objects, a method for ablating a tumor in a body while protecting a nearby structure from the effects of the ablation comprises the steps of inserting an ablation device to a location in the body proximate the tumor, interposing an ablation shield between the tumor and the nearby critical structure and then activating one or more ablation sources of the ablation device to ablate the tumor while the nearby structure is shielded by the ablation shield. Preferably, the ablation procedure is performed while imaging at least a region including and surrounding the tumor so that the ablation device can be guided toward the tumor based on the imaging. The ablation shield can also be guided percutaneously between the nearby structure and the tumor based on the imaging.

If the ablation is a radiofrequency ablation, then the ablation sources are a plurality of wires which are activated to emit radio-frequency current from their tips to create heat to ablate the tumor.

In one embodiment, the ablation shield is an at least partially inflatable balloon which may be inflated with a fluid after the balloon is interposed between the tumor and the nearby structure and prior to activation of the ablation source(s). Inflation of the balloon causes an increase in the distance between the tumor and the nearby structure and also forms a barrier which may be designed to prevent the transmission of heat generated by the radiofrequency current to the nearby structure. In the latter case, it would be beneficial to form the balloon from a shielding material which counteracts the effects of the radiofrequency current, i.e., as a heat shield which could be made from a heat-absorbing material.

In another embodiment, the ablation shield is a fan retractor having an expandable fan portion folded upon interposition of the fan retractor between the tumor and the nearby structure. The fan portion is expanded after the fan retractor is interposed between the tumor and the nearby structure and prior to activation of the ablation source(s). The orientation of the fan retractor can be selected such that the fan portion expands either to push the nearby structure away from the tumor (in which case the distance between the nearby structure and tumor reduces the impact of the ablation source(s) on the nearby structure) or to cause a substantial part of the fan portion to be present between the tumor and the nearby structure (in which case, the fan material acts as a shield to reduce the impact of the ablation source(s) on the nearby structure). The fan may be partially composed of an x-ray attenuating material that would shield from radiation. This may be appropriate in brachytherapy applications in the breast or prostate for example.

A method for treating a tumor requiring multiple, sequential treatment which may use the ablation shield described above comprising the steps of performing a first treatment on the tumor (interposing the ablation shield between the tumor and the nearby structures if desired), marking the area of the tumor treated during the first treatment and performing at least one subsequent treatment on the tumor based the marked area of the tumor. If the treatments are radiofrequency ablations, marking the area of the tumor may entail placing a radio-opaque material at a location at ends of wires of a needle probe used in the radiofrequency ablations.

A method for differentiating between instruments used in surgery in accordance with the invention comprises the step of providing a plurality of instruments used for surgery with a different signature and enabling visibility of the signatures during imaging performed during the surgery. To this end, each instrument may incorporate with the same signature in multiple signature types such that the same signature is visible for imaging for multiple modalities.

### BRIEF DESCRIPTION OF THE DRAWINGS

The invention, together with further objects and advantages hereof, may best be understood by reference to the following description taken in conjunction with the accompanying drawings, wherein like reference numerals identify like elements and wherein:

FIG. 1 is a schematic view showing placement of a first embodiment of a shield between a tumor on a liver being treated and a portion of a normal intestine for use in a method in accordance with the invention.

FIG. 2 is a front view of a second embodiment of a shield in accordance with the invention.

FIG. 3 is a front view of the shield shown in FIG. 2 in a position during use in a method in accordance with the invention.

FIG. 4A is a front view of the shield shown in FIG. 2 in a position during a first manner of use in which a material is interposed between a tumor and healthy, nearby structure in a method in accordance with the invention.

FIG. 4B is a front view of the shield shown in FIG. 2 in a position during a second manner of use in which it pushes healthy structure away from a tumor being treated in a method in accordance with the invention.

FIG. 5 is a view of a tumor being treated.

FIG. 6 is a view showing the application of a marking agent to indicate a treated portion of the tumor.

FIG. 7 is a view showing the tumor of FIG. 5 after multiple applications of a marking agent.

FIG. 8 is a view of a first instrument with a signature in accordance with the invention when viewed on a screen of an imaging device.

FIG. 9 is a view of a second instrument with a signature in accordance with the invention when viewed on a screen of an imaging device.

FIG. 10 shows a biopsy needle used for percutaneous electrocautery.

FIG. 11 shows an instrument for use in appendectomies in accordance with the invention.

FIG. 12 shows the instrument of FIG. 11 in use for an appendectomy.

FIGS. 13 and 14 show a side view of the operative use of the instrument in FIG. 11.

FIG. 15 shows a view of the removal of the cut portion of the appendix.

FIG. 16 shows a resector and/or morcellator in accordance with the invention.

FIGS. 17 and 18 show operative positions of use of the resector and/or morcellator shown in FIG. 16.

### DETAILED DESCRIPTION OF THE INVENTION

Described below are several innovations for performing surgery and more particularly for performing percutaneous surgery in conjunction with imaging of the surgical area. Each of these innovations can be used independently of one another or in combination with one another.

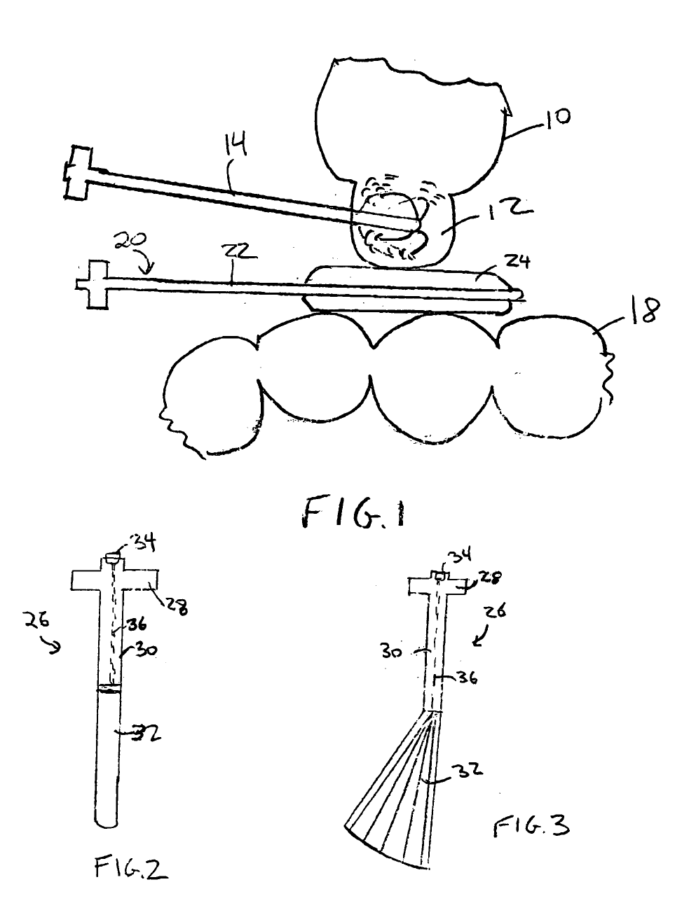

Referring first to FIG. 1, a method for ablating a tumor will be described in connection with several embodiments of an apparatus in accordance with the invention in which the ablation source used to ablate the tumor is a plurality of radiofrequency current-generating sources. It should be understood that other ablation and radiation sources can also be used in the invention.

As shown in FIG. 1, it is desired to ablate a tumor 12 on a liver 10, for example. To this end, a needle probe 14 with a plastic insulated shaft is inserted into the tumor 12, preferably percutaneously with imaging guidance. When the surgeon presses a button on the needle probe 14, wires 16 similar to the backbone of an umbrella shoot out and cover a portion or all of the tumor 12. The wires 16 are then activated so that alternating radio-frequency current is generated at the tips of the wires 14 and agitates ions in the tissue surrounding the needle probe 14, creating frictional heat to heat the tumor 12.

Prior to the activation of the wires 16, it is determined whether there is/are tissue or organs (represented by a portion of the intestines 18) proximate to the tumor 12 which would be adversely affected by the heat created by the radio-frequency current applied to the wires 16 and thus should be protected therefrom. If so, an ablation shield 20 is interposed percutaneously between the tumor 12 and the intestines 20 to prevent the transmission of heat from the tips of the wires 14 to the intestines 18.

The ablation shield 20 may take various forms. In the form illustrated in FIG. 1, the ablation shield 20 is a balloon catheter 22, at least an interior portion 24 of which is inflatable with a fluid. The balloon catheter 22 usually enters the body in a non-inflated state and is guided, using imaging techniques, between the tumor 12 and the intestines 18 and then a fluid is directed into the interior portion 24.

The fluid may be a gas, such as air and carbon dioxide, or a non-conducting liquid such as deionized water. The fluid used depends in part on the effect being applied to the tumor 12, with the fluid preferably being one which is designed to counter or prevent the transmission of the effect being applied to the tumor 12. For example, for radiofrequency ablation, the effect is one of heat so that the fluid in the balloon catheter 22 would preferably be one which prevents the transmission of heat therethrough. If the effect is ultrasound, i.e., an ultrasonic procedure is being applied to ablate the tumor 12, then the fluid should preferably be a material which impedes the transmission of ultrasonic energy. Thus, the fluid may be selected based on the type of ablation source, e.g., radiofrequency energy, ultrasonic energy, etc.

In addition to containing a substance that counters the effect being applied to the tumor 12, the balloon catheter 22 also serves to increase the distance between the tumor 12 and the intestines 18. This is highly advantageous for treatments which have a reduced effect over distance. For example, heat dissipates in a direction away from the source so that by increasing the distance between the heat source (the tips of the wires 16) and the intestines 18, the effect of the heat is reduced irrespective of the presence of any fluid in the balloon catheter 22. In this regard, the balloon catheter 22 can be inflated to have various diameters and thereby enable a variation in the extent to which the intestines 18 are separated from the tumor 12.

Moreover, various sizes of balloon catheters 22 may be used, e.g., an 8 mm balloon and a 22 mm balloon. The larger balloon can contain more fluid than the smaller balloon and have a larger inflatable interior enabling a larger separation between the tumor 12 and the intestines 18 than the smaller balloon. Indeed, it has been found that the larger balloon results in a greater temperature reduction between a tumor 12 and the intestines 18 than the smaller balloon.

Furthermore, the balloon catheter 22 may be formed from a material which serves as a shielding material to protect from ablation sources. For radiofrequency ablation, the material of the balloon catheter 22 would be non-absorbent of radiofrequency energy, In the case of thermal ablation, the balloon 22 may include or be made of a material that would have a protective heat capacity. In the case of radiation protection, the balloon may include or be made of lead. These radioactive shields may also be placed prior to external radiation treatment.

An added feature of the balloon catheter 22 would be to make the material of the balloon catheter 22 of a firm plastic material or non-puncturable material so that it would resist bursting if a needle probe accidentally contacted it.

After the radio-frequency energy is applied to the wires 16 and the ablation is complete, the balloon catheter 22 is deflated and withdrawn from the body and the needle probe 14 is also withdrawn.

An alternative ablation shield 20 is a fan retractor 26 as shown in FIGS. 2 and 3. The fan retractor 26 has a body having a head 28 and a shaft 30 and is inserted percutaneously into the body shaft first. The shaft 30 includes an expandable fan portion 32 which is folded upon interposition of the fan retractor 26 between the tumor 12 and the nearby intestines 18 (see FIG. 2). Once the fan retractor 26 is positioned between the tumor 12 and the nearby intestines 18, a button 34 on the head 28 is pressed releasing the hold on the fan portion 32 and causing the fan portion 32 to expand (see FIG. 3). The mechanism causing the hold on the fan portion 32 until pressing of the button 34 would be readily constructable by one skilled in the art and would include a coupling 36 as shown in dotted lines in FIGS. 2 and 3 which converts the downward pressure on the button 34 into a movement releasing the restraint on the fan portion 32.

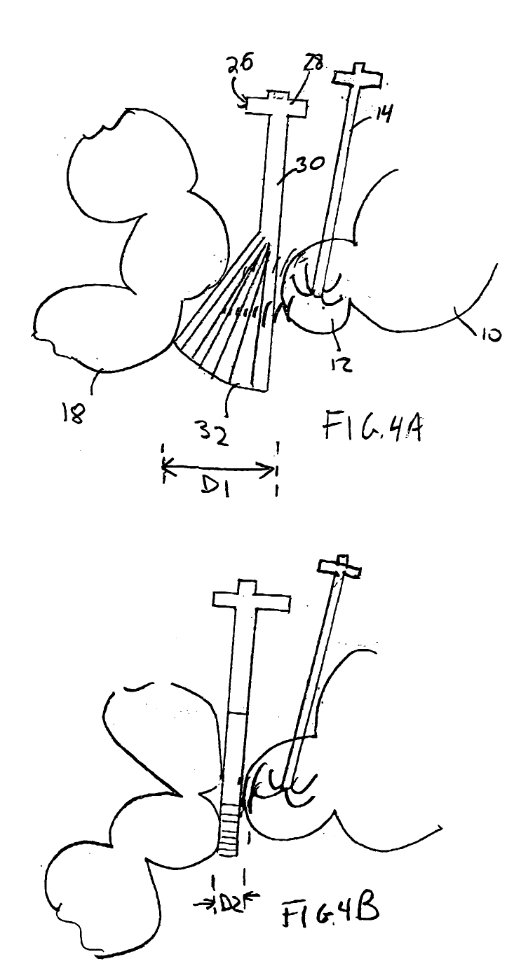

Expansion of the fan portion 32 causes either the interposition of a substantial portion of the fan portion 32 between the tumor 12 and the intestines 18 when the fan portion 32 expands at an angle to a plane defined by the tumor 12 and the intestines 18 (FIG. 4A) or a forced separation of the intestines 18 away from the tumor 12 when the fan portion 32 expands against the intestines 18 in the plane defined by the tumor 12 and the intestines 18 (see FIG. 4B). Thus, the retractor fan 26 can be oriented to either provide a physical shield between the tumor 12 and the intestines 18 (FIG. 4A) or separation between the intestines 18 and the tumor 12 (FIG. 4B). The use of the fan retractor 26 may depend on the type of ablation.

Note that the distance between the intestines 18 and the tumor 12 (D1) is greater in FIG. 4A when the retractor fan 26 is used to separate the intestines 18 from the tumor 12 than the distance (D2) as shown in FIG. 4B when the retractor fan 26 is used as a shield between the intestines 18 and the tumor 12. Separation of the intestines 18 from the tumor 12 (FIG. 4A) reduces the effect of the ablation source when the effect decreases with distance, such as heat.

When used as a shield (FIG. 4B), the material of the fan portion 32 of the retractor fan 26 can be selected to counteract the effects of the ablation source, e.g., the tips of the wires 16 which emit radiofrequency energy. For example, in the case of thermal ablation, the fan portion 32 may include or be made of a material that would have a protective heat capacity. In the case of radiation ablation, the fan portion 32 may be made of a material such as lead or include lead. In the case of microwave energy used for ablation, the fan portion 32 may be made of an electromagnetic shielding material.

Referring now to FIGS. 5-7, an issue in the percutaneous thermal ablation of the tumor may arise if the tumor 40 is larger than the effective area of the ablation source. In this case, multiple ablations must be performed, each effective on a portion of the tumor 40. The combined effect of the multiple ablations will hopefully encompass the entire tumor 40. However, for a subsequent ablation of a portion of the tumor 40, it is not easy to ascertain which portion of the tumor 40 has already been ablated. This issue also arises for interstitial radiation procedures.

To facilitate identification of a portion of a tumor 40 which has already been ablated so that a subsequent ablation can be performed on a non-ablated portion of the tumor 40, possibly with a slight overlap with the ablated portion, the present invention includes a marking or labeling system in which after ablation of a portion of the tumor 40, that portion is marked. In this manner, in spite of the performance of multiple ablations, there is no gap between treatment zones.

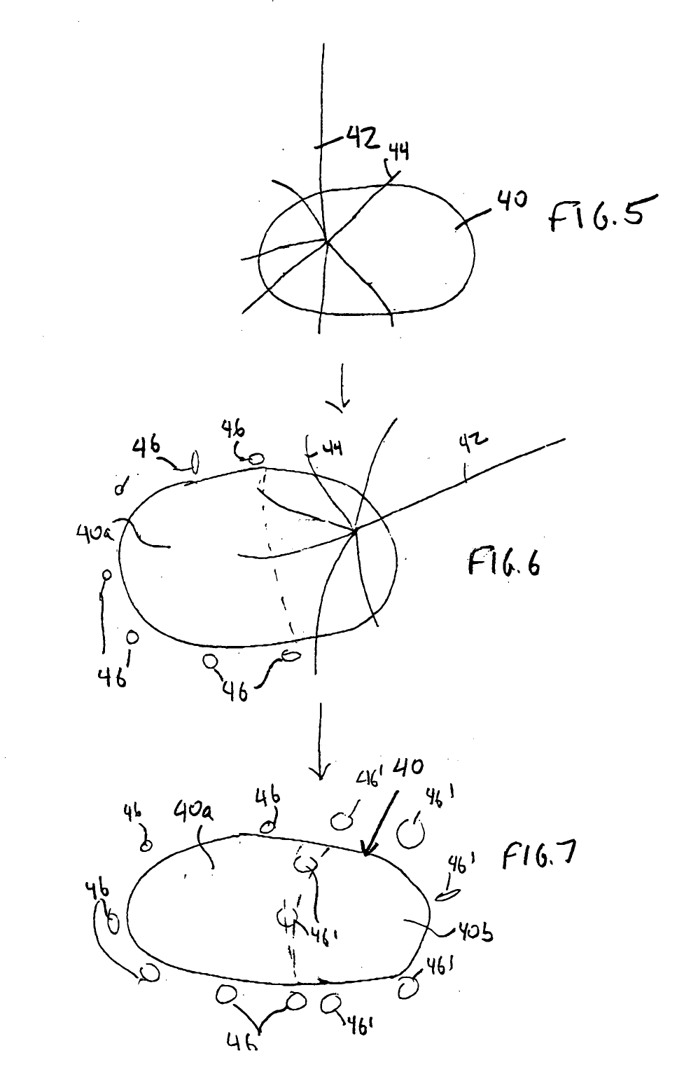

As shown in FIG. 5, the tumor 40 is larger than the effective area of the ablation source, i.e., the radiofrequency current generated by the tips of wires 44 from the needle probe 42. Thus, a first ablation is performed in which the left side of the tumor 40 is ablated (portion 40a). After the radiofrequency current is stopped, a marker 46 is released at the locations of the tips of the wires 44 (see FIG. 6). The markers 46 may be a radio-opaque glue material such as a mixture of methylmethacrylate and lipiodol that is released through the ablation needle tines. The markers 46 may alternatively be released from a separate surgical instrument inserted percutaneously into the body, i.e., a needle or the like. That is, while imaging the area of the tumor 40, a needle filled with a marking agent can be introduced into the body and pressed to release a drop of marking agent at the locations of the tips of the wires 44.

The marking agent should be visible to the imaging procedure. Thus, a magnetic imaging procedure would be used in conjunction with a magnetic material such as a metal fragment or staple or a vitamin E containing mixture. An x-ray imaging procedure would be used in conjunction with a radio-opaque material, such as a barium or iodinated dye or glue.

The needle probe 42 is then removed and re-positioned to ablate the non-ablated portion of the tumor 40 (portion 40b). Ablation is performed and then the marking agent is again placed at the location of the tips of the wires 44 of the needle probe 42 (FIG. 7). The image of the tumor 40 is then viewed to confirm that the entire tumor 40 is ablated by the two ablation procedures.

By marking or labeling the ablated portion of a tumor, it is easy to identify the non-ablated portion of the tumor and thereby facilitate placement of the needle probe using imaging.

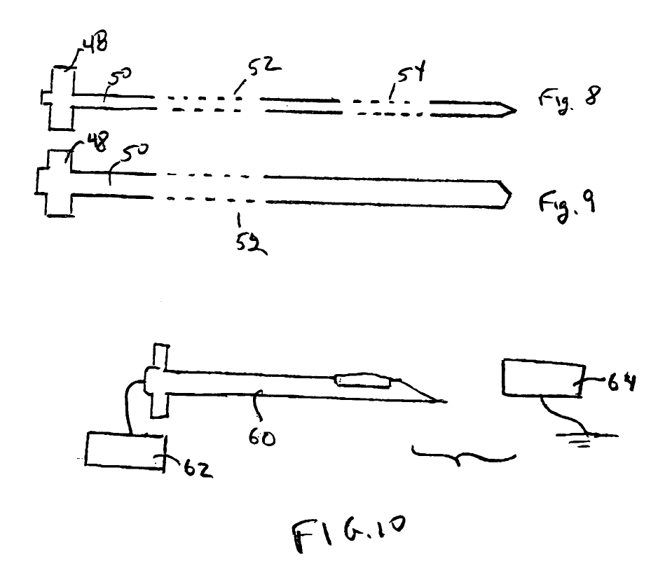

Referring now to FIGS. 8 and 9, it is often necessary and desirable to know the position of specific instruments in a body during surgery when multiple instructions are being used. Although the identity of the instruments is readily ascertainable when the instruments are outside of the body, when inside the body, it is sometimes difficult to tell which instrument is which.

To facilitate identification of instruments when used during surgery in which the surgical area is being imaged, the present invention provides for the incorporation of an imaging signature into surgical instruments so that when imaged, each instrument provides a signature which indicates the type and identity of the instrument. Each different instrument has a unique signature.

The “imaging signature” may be different depending on the imaging modality being used (e.g., ultrasound, CT, MR, PET). Multiple signature types may be applied to an instrument so that the instrument may keep its signature regardless of the imaging modality being used.

In one embodiment, an x-ray or CT imaging signature may be accomplished by incorporating material of reduced radiodensity within the shaft 50 of the instrument 48. As shown in FIGS. 8 and 9, the material of reduced radiodensity at locations 52 and 54. This would create a “dashed” appearance. The instrument shown in FIG. 8 has two dashed areas 52,54 and thus would be known to the surgeon as being different from the instrument shown in FIG. 9. Of course, the surgeon has to known the signature of the instruments along with the identification of the instrument, i.e., he or she has to known that two dashed areas 52,54 exemplifies a thin needle probe (FIG. 8) where a single dashed area 52 exemplifies a thick needle probe (FIG. 9).

Other means might be creating different thicknesses of radiodense material. In ultrasound, the signatures would be material with different echogenicities.

Referring now to FIG. 10, one of the concerns of performing “surgery” percutaneously is control of bleeding. Electrocautery is used in surgery to coagulate blood and stop bleeding. Electrocautery can be incorporated into percutaneous tools such as biopsy needles and ablation tools.

FIG. 10 shows a biopsy needle 60 connected to an electrical source 62. A grounding pad 64 is also provided to attach to the patient to ground them and prevent electrical shock. In use, the biopsy needle 60 is inserted into the body to cut a portion of the body for analysis external to the body. After the portion of the body is cut, the part of the body from which the portion was cut may bleed. To cauterize this portion, the tip 66 of the biopsy needle 60 may be placed close to the bleeding site and the electrical source 62 activated. The activation of the electrical source 62 may be performed immediately after the cut portion is received by the biopsy needle 60. It is important not to heat the specimen such that it becomes uninterpretable.

By coupling a cauterization function to a biopsy needle (or to an ablation tool which would be the same manner), an efficient and safe percutaneous electrocautery can be achieved.

The biopsy needle 60 may be provided with an imaging signature as discussed above with reference to FIGS. 8 and 9.

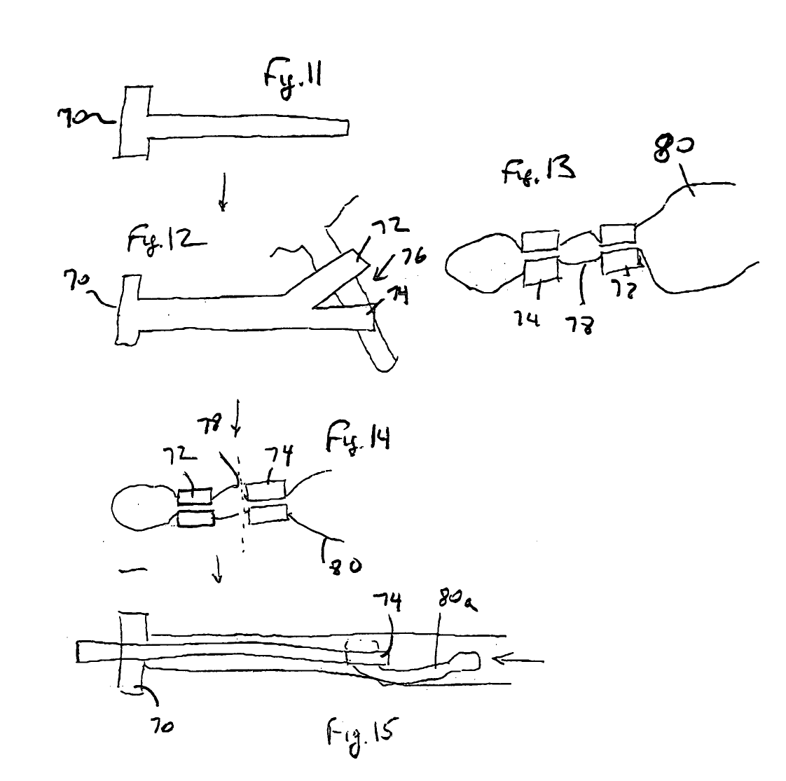

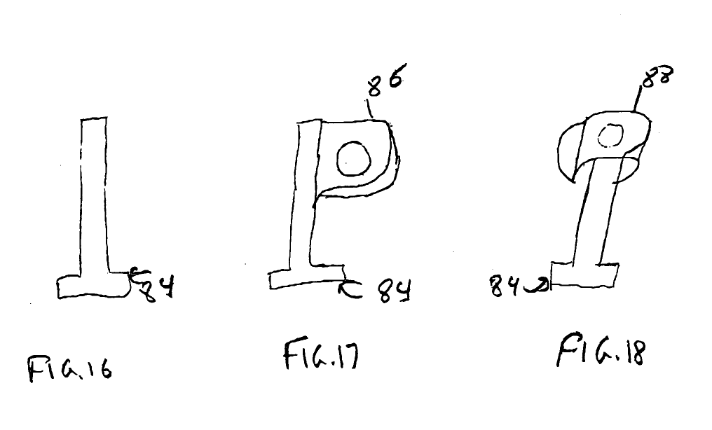

Referring now to FIGS. 11-15, in order to perform an appendectomy percutaneously, an instrument 70 in accordance with the invention is provided with two prongs 72, 74 at a proximal end defining a jaw 76 which surrounds the cutting site 78 of the target appendix 80. The instrument 70 is inserted percutaneously into the body and manipulated to cause two portions of the appendix 80 to be grasped, one by each prong 72, 74 (as shown in FIG. 13). Thereafter, the appendix 80 is cut at the cutting site 78 (FIG. 14). The cut portion of the appendix 80a may be removed when one prong 74 is opened (see FIG. 15).

The construction of the instrument 70 with two adjustable prongs 72,74, independently adjustable by manipulation of a component at the distal end, can be readily obtained by one skilled in the art without undue experimentation.

The instrument 70 may be provided with an imaging signature as discussed above with reference to FIGS. 8 and 9.

Referring now to FIGS. 16-18, a percutaneous resector and/or morcellator 84 is shown for use when tissue needs to be removed from the body. The resector 84 includes a wire 86 with a neck such as a butterfly neck. The resector 84 is manipulated such that the wire 86 surrounds or circumscribes the portion of the body to be excised (FIG. 17). The wire 86 is then heated to cause the portion to be excise and cut the tissue in a loop. The resector 84 also includes a net 88 which captures the excised portion of the body (FIG. 18).

The resector 84 may be provided with an imaging signature as discussed above with reference to FIGS. 8 and 9.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

I claim:

1. A method for ablating or irradiating a tumor in a body while protecting a nearby structure from the effects of the ablation, comprising the steps of:
inserting an ablation device to a location in the body proximate the tumor, the ablation device having at least one ablation source;
interposing an ablation shield between the tumor and the nearby structure; and then activating the ablation source to ablate the tumor while the nearby structure is shielded by the ablation shield.

2. The method of claim 1, further comprising the steps of:
imaging at least a region including and surrounding the tumor; and
guiding the ablation device toward the tumor based on the imaging.

3. The method of claim 1, wherein at least one ablation source is a plurality of wires, the wires being activated to emit radio-frequency current from their tips to create heat to ablate the tumor.

4. The method of claim 1, wherein the ablation shield is interposed percutaneously.

5. The method of claim 1, further comprising the steps of:
imaging at least a region including and surrounding the tumor and the nearby structure; and
guiding the ablation shield to a position between the tumor and the nearby structure based on the imaging.

6. The method of claim 1, further comprising the steps of:
imaging at least a region including and surrounding the tumor and the nearby structure; guiding the ablation device toward the tumor based on the imaging; and
guiding the ablation shield to a position between the tumor and the nearby structure based on the imaging.

7. The method of claim 1, wherein the ablation shield is a balloon.

8. The method of claim 7, further comprising the step of inflating the balloon with a fluid after the balloon is interposed between the tumor and the nearby structure and prior to activation of the at least one ablation source.

9. The method of claim 8, further comprising the step of selecting the fluid to inflate the balloon from a group consisting of air, carbon dioxide and deionized water.

10. The method of claim 8, further comprising the step of selecting the fluid based on the type of ablation source.

11. The method of claim 1, further comprising the step of constructing the ablation shield to increase a distance between the tumor and the nearby structure when interposed therebetween.

12. The method of claim 1, further comprising the step of constructing the ablation shield from a material which serves as a shielding material to counteract the effects of the at least one ablation source.

13. The method of claim 1, wherein the ablation shield is a fan retractor having an expandable fan portion folded upon interposition of the fan retractor between the tumor and the nearby structure, further comprising the step of expanding the fan portion after the fan retractor is interposed between the tumor and the nearby structure and prior to activation of the at least one ablation source.

14. The method of claim 13, further comprising the step of selecting the orientation of the fan retractor such that the fan portion expands either to push the nearby structure away from the tumor or to cause a substantial part of the fan portion to be present between the tumor and the nearby structure.

15. The method of claim 13, wherein the fan portion expands to causes a substantial part of the fan portion to be present between the tumor and the nearby structure, further comprising the step of selecting the material of the fan portion to counteract the effects of the at least one ablation source.

16. A method for treating a tumor requiring multiple, sequential treatment, comprising the steps of:
performing a first treatment on the tumor;
marking the area of the tumor treated during the first treatment; and
performing at least one subsequent treatment on the tumor based the marked area of the tumor.

17. The method of claim 16, wherein the treatments performed on the tumor are radiofrequency ablations, the step of marking the area of the tumor comprising the step of placing a radio-opaque material at a location at ends of wires of a needle probe used in the radiofrequency ablations.

18. A method for differentiating between instruments used in surgery, comprising the steps of:
providing a plurality of instruments used for surgery with a different signature; and
enabling visibility of the signatures during imaging performed during the surgery.

19. The method of claim 18, further comprising the step of incorporating each instrument with the same signature in multiple signature types such that the same signature is visible for imaging for multiple modalities.

20. A method for biopsying an internal portion of the body, comprising the steps of:
inserting a biopsy needle into the body;
cutting the internal portion of the body with the biopsy needle;
positioning a tip of the biopsy needle near to the biopsy site;
grounding the body; and
activating an electrical source to apply electricity to at least a tip of the biopsy needle such that the biopsy site is cauterized.
